(19) United States
(12) Patent Application Publication
(10) Pub. No.: US 2026/0311856 A1
(43) Pub. Date: Oct. 8, 2026

(54) MACHINE-LEARNING ARCHITECTURES FOR AUTOMATED SYSTEM OPERATIONS

(71) Applicant: MYRIAD SYSTEMS INC., Valhalla, NY (US)

(72) Inventors: Jeremy SHINER, Valhalla, NY (US); Enis TAHIRAJ, Valhalla, NY (US)

(73) Assignee: MYRIAD SYSTEMS INC., Valhalla, NY (US)

(21) Appl. No.: 19/637,913

(22) Filed: Apr. 2, 2026

Related U.S. Application Data
(60) Provisional application No. 63/783,143, filed on Apr. 3, 2025.

(51) Int. Cl. G16H 40/20; G06N 20/00

(52) U.S. Cl. CPC G16H 40/20; G06N 20/00

(57) ABSTRACT

Embodiments include systems and methods for identifying routine provider operations or actions and suggesting which routine provider action may be automated provider actions using machine learning. A feature vector represents provider actions, and actions satisfying a regularity threshold are identified as routine. For each routine action, a suggested candidate action score is generated based on the feature vector and improvement features. A candidate action is suggested for automation if its score meets an automation threshold. The system can display the candidate automated action on a user interface and train a machine-learning model to refine the candidate action score. Additionally, routine action scores are generated to indicate the likelihood of actions being routine, aiding in accurate automation decisions.

### CROSS-REFERENCE TO RELATED APPLICATION

This application claims priority to and the benefit of U.S. Provisional Application No. 63/783,143, filed Apr. 3, 2025, which is incorporated by reference in its entirety.

### TECHNICAL FIELD

The present disclosure generally relates to systems and methods for automated clinical practice management. More specifically, embodiments include machine-learning architectures to improve the accuracy and efficiency of capturing and generating practice management operations for healthcare providers.

### BACKGROUND

Existing technologies for managing healthcare practices have several technological shortcomings. For instance, conventional clinical practice management systems, including clinical payment systems and/or revenue cycle management systems typically do not leverage advanced technologies like AI, Optical Character Recognition (OCR), and/or automated reporting to streamline the payment process. This can lead to manual errors, delays, and a lack of transparency in cost estimation and explanation. This can impact, for example, convention patient management and appointment handling or check-in systems, which often require manual input and lack efficiency.

Conventional practice management systems generate and handle patient data, such that the processes rely on patient profiles gathered from manual data entry or other systems. However, traditional patient profiles often lack comprehensive front-office and back-office financial management information as integrated with traditional Electronic Health Records (EHR) clinical information, leading to fragmented and incomplete patient information. This fragmentation can result in inefficiencies and errors in patient care and billing.

Some conventional systems may attempt to employ artificial intelligence (AI) or machine-learning model (ML) for improving aspects of practice management systems. Yet, a significant shortcoming in these prior attempts is the accuracy and reliability of the AI/ML models in interpreting complex insurance profiles. For instance, insurance policies often have intricate terms and conditions, varying deductibles, co-payments, and co-insurances that can be challenging for AI/ML models to accurately parse and understand. This complexity can lead to errors in determining patient fees or treatment eligibility, resulting in incorrect or misleading information being provided to providers or patients. As such, conventional systems provide unstructured eligibility and insurance coverage data and leave the calculation to the practice, leading to widespread mistakes, wasted time, and ultimately resulting in patient dissatisfaction and confusion.

Another issue with prior approaches to implement AI/ML solutions is that such approaches lack integration between the AI/ML models and existing healthcare provider systems and databases. Ensuring seamless integration with Electronic Health Records (EHR) and insurance databases is crucial for the AI/ML models to access up-to-date and comprehensive patient information. However, this integration can be technically challenging and may require significant resources and time to implement effectively.

Additionally, the AI/ML models need to be continuously updated and trained with new data to maintain their accuracy and relevance. Insurance policies and healthcare regulations frequently change, and the models must adapt to these changes to provide accurate estimates. This ongoing maintenance can be resource-intensive and may pose a challenge for healthcare providers.

Another challenge of explaining the AI/ML-generated estimates to patients in a clear and understandable manner. Patients may find it difficult to comprehend the itemized financial responsibilities and the explanation of benefits generated by the AI/ML models. Ensuring that the information is presented in a user-friendly and transparent way is essential to avoid confusion and build trust with patients.

### SUMMARY

Disclosed herein are systems and methods capable of addressing the above-described shortcomings and may also provide any number of additional or alternative benefits and advantages. Embodiments may include computing systems for estimating patient liability. Traditional methods often involve manual calculations, leading to errors and inefficiencies. Embodiments may implement machine-learning models trained to accurately predict patient liabilities, reducing manual errors and enhancing transparency in cost estimations. Embodiments may include computing systems and machine-learning models trained for confirming patient eligibility for certain clinical procedures. Existing systems struggle with interpreting complex insurance terms and conditions. By employing the machine-learning models described herein, the embodiments described herein may address the inaccuracies in determining patient eligibility and provides reliable information, thereby improving patient care and billing accuracy.

Embodiments may include computing systems for a provider management software tool. Conventional practice management systems often lack efficiency in handling routine pattern operations. Embodiments may implement machine-learning models trained to identify routine (or pattern) management tasks and suggests automated operations of software routines (or functions), streamlining operations, and reducing the administrative burden on healthcare providers.

Embodiments may include computing systems for an integrated patient profile evaluation system, including credit reporting service integration with a practice management system. Fragmented patient information can lead to incomplete profiles and inefficiencies in patient care. This embodiment integrates patient profiles with comprehensive data from credit reporting services and practice management systems, providing a holistic view of patient information and enhancing the quality of care.

Embodiments may include a computer-implemented method for identifying automated operations for management systems, the method including: obtaining, by a computer, management operation data for one or more management operation executed by a management application; for each management operation, extracting, by the computer, a feature vector representing the management operation of the management operation data; identifying, by the computer, a management operation as a pattern operation based on the feature vector representing the management operation and a number of instances of the management operation satisfying a regularity threshold number of instances; for each pattern operation, generating, by the computer, a candidate operation score for the pattern operation based upon the feature vector representing one or more features extracted for the management operation identified as the pattern operation and a set of improvement features representing improved management operations; and generating, by the computer, an instruction for the management application to execute the pattern operation as a candidate automated operation in response to the computer determining that the candidate operation score satisfies an automation threshold.

The method may further include generating, by the computer, a message indicating the candidate automated operation for display at user interface of a management computing device.

The method may further include training, by the computer, a candidate automation machine-learning model to generate the candidate operation score.

When identifying the management operation as the pattern operation, the method may further include generating, by the computer, a pattern operation score based on the feature vector and a set of expected pattern features representing one or more expected pattern operation, the pattern operation score representing a likelihood of the management operation being the pattern operation. The computer may determine that the candidate operation score satisfies a predefined pattern threshold and the regularity threshold.

The method may further include training, by the computer, a candidate automation machine-learning model to generate the pattern operation score. The management operation data may include patient profile data obtained by the computer from an external server.

Embodiments may include a system for identifying automated operations for management systems, the system including: a computer including at least one processor, configured to: obtain, from a database, management operation data for one or more management operations executed by a management application; for each management operation, extract a feature vector representing the management operation of the management operation data; identify a management operation as a pattern operation based on the feature vector representing the management operation and a number of instances of the management operation satisfying a regularity threshold number of instances; for each pattern operation, generate a candidate operation score for the pattern operation based upon the feature vector representing one or more features extracted for the management operation identified as the pattern operation and a set of improvement features representing improved management operations; and generate an instruction for the management application to execute the pattern operation as a candidate automated operation in response to determining that the candidate operation score satisfies an automation threshold.

The computer may be further configured to generate a message indicating the candidate automated operation for display at user interface of a management computing device.

The computer may be further configured to train a candidate automation machine-learning model to generate the candidate operation score.

When identifying the management operation as the pattern operation, the computer may be further configured to generate a pattern operation score based on the feature vector and a set of expected pattern features representing one or more expected pattern operations, the pattern operation score representing a likelihood of the management operation being the pattern operation. The computer may determine that the candidate operation score satisfies a predefined pattern threshold and the regularity threshold.

The computer may be further configured to train a candidate automation machine-learning model to generate the pattern operation score. The management operation data may include patient profile data obtained by the computer from an external server.

Embodiments may include a non-transitory machine-readable storage configured to machine-readable instructions for identifying candidate automated operations for management systems that, when executed by at least one processor, instructs the at least one processor to: obtain, from a database, management operation data for one or more management operations executed by a management application; for each management operation, extract a feature vector representing the management operation of the management operation data; identify a management operation as a pattern operation based on the feature vector representing the management operation and a number of instances of the management operation satisfying a regularity threshold number of instances; for each pattern operation, generate a candidate operation score for the pattern operation based upon the feature vector representing one or more features extracted for the management operation identified as the pattern operation and a set of improvement features representing improved management operations; and generate an instruction for the management application to execute the pattern operation as a candidate automated operation in response to determining that the candidate operation score satisfies an automation threshold.

The instructions may further instruct the at least one processor to generate a message indicating the candidate automated operation for display at user interface of a management computing device.

The instructions may further instruct the at least one processor to train a candidate automation machine-learning model to generate the candidate operation score.

When identifying the management operation as the pattern operation, the instructions may further instruct the at least one processor to generate a pattern operation score based on the feature vector and a set of expected pattern features representing one or more expected pattern operation, the pattern operation score representing a likelihood of the management operation being the pattern operation. The at least one processor may determine that the candidate operation score satisfies a predefined pattern threshold and the regularity threshold. The instructions may instruct the at least one processor to train a candidate automation machine-learning model to generate the pattern operation score.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 depicts the various device components of the system in a networked environment, according to some embodiments.

FIG. 2 shows a flowchart of operations of a computer-implemented method for identifying and suggesting automated candidate operations or actions, according to some embodiments.

FIG. 3 shows a flowchart of operations of a computer-implemented method for determining or predicting patient eligibility for a healthcare service, according to some embodiments.

FIG. 4 shows a flowchart of operations of a computer-implemented method for generating an estimated patient liability using one or more machine-learning models of a machine-learning architecture, according to some embodiments.

FIG. 5 shows a flowchart of operations of another embodiment for a computer-implemented method for generating an estimated patient liability using a patient liability amount using third-party patient data hosted at a third-party database, according to some embodiments.

### DETAILED DESCRIPTION

Reference will now be made to the illustrative embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to a person skilled in the relevant art and having possession of this disclosure, are to be considered within the scope of the invention.

Embodiments may include computing systems for estimating patient liability. A machine-learning model may be trained to ingest a patient's insurance profile and generate an estimate of the patient's financial responsibility (e.g., deductibles, co-payments, co-insurances). Based upon features of the patient's insurance profile, the AI/ML may generate a report that includes, for example, itemized patient financial responsibilities and an explanation of benefits.

Embodiments may include computing systems for confirming patient eligibility for certain clinical procedures. A machine-learning model may be trained to ingest a patient's insurance profile and determines whether the patient is eligible for certain treatments or benefits based on features of the patient's insurance profile.

Embodiments may include computing systems for a provider management software tool. In some embodiments, the provider management software tool is implemented as a management system comprising one or more computing devices executing a management application to generate, store, transmit, and/or display practice management information and to execute management operations. A management computing device may include any computing device executing the management application and/or displaying outputs generated by the management application. A machine-learning model may be trained to identify routine provider management operations (sometimes referred to as management actions) and to identify which routine or regimented operations (sometimes referred to as regimented actions) are candidates for automation. The machine-learning model may be trained to suggest automated operations (sometimes referred to as automated actions) from the identified routine operations (sometimes referred to as routine actions), such as operations that address insurance and patient balances (e.g., sending invoices, charging patient accounts). Users of a provider management system or practice management application may implement the suggested automated candidate operations (sometimes referred to as candidate operations or candidate actions) or the software may automatically automate the suggested automated operations, such as certain financial management operations.

Embodiments may include computing systems for integrated patient profile evaluation system, including credit reporting service integration with a practice management system. For example, a computing system of a provider or practice management application may transmit a soft credit checks on a patient to a third-party server hosting a credit reporting service. A computer may execute machine-learning model trained to, for example, perform the patient eligibility check, patient liability determination, or suggest one or more payment plan options, including down payments and payment plan lengths based upon attributes and history of the patient, among other operations.

Embodiments may implement a self-service kiosk device located at a care provider's office. The kiosk includes computing device programmed and configured to function as a self-service terminal and/or a payment terminal for patients during patient visits. The kiosk may be wired or wirelessly networked to other computers of the care provider, which includes administrative software that can control or manage the kiosk. Conventionally, medical offices will offer a self-service terminal to navigate a building or appointments and/or a payment terminal, separate from the self-service terminal. Embodiments of the self-service kiosk device described herein combine these functions. Patients can use the self-service kiosk to, for example, sign-in and check-in for an appointment, enter patient-supplied data into patient forms, and make payments, among other features. In some implementations, the self-service kiosk can also seamlessly transition between self-service and staff-controlled administration operating modes. For instance, the provider staff of the medical office can remotely control the self-service kiosk screen to, for example, push or present patient forms at the user interface for the patient to complete or enter payment information for payment requests, among other administrative operations.

### Example System Components

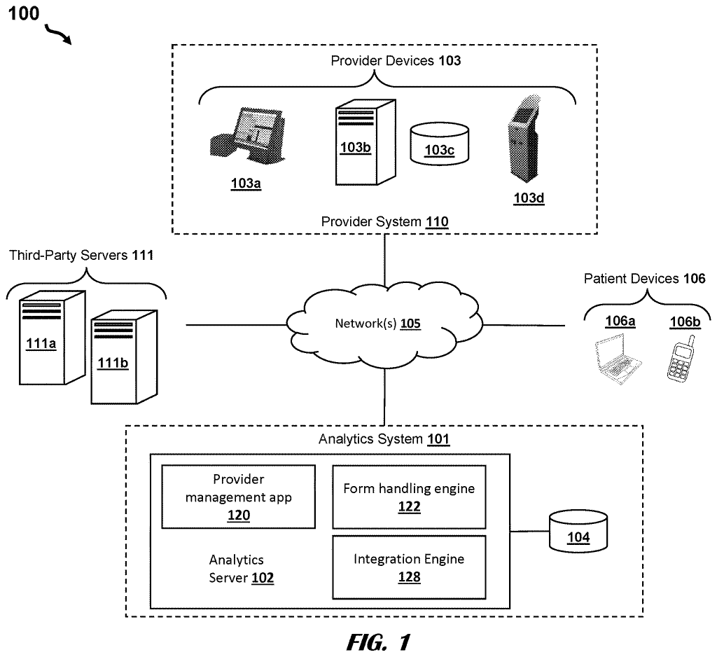

FIG. 1 shows components of a system 100 for clinical practice management using one or more machine-learning architectures having one or more machine-learning models for integrating various types of data and data sources. FIG. 1 depicts the various device components of the system 100 in a networked environment as well as an example collection of executable software engines of analytics servers 102.

Embodiments may comprise additional or alternative components or omit certain components from those of FIG. 1, and still fall within the scope of this disclosure. It may be common, for example, to include provider systems and third-party systems with any number of provider devices 103 and third-party servers 111. Embodiments may include or otherwise implement any number of devices capable of performing the various features and tasks described herein. For example, FIG. 1 depicts the analytics server 102 as a distinct computing device from the analytics database 104. In some embodiments, an analytics database 104 may be integrated into the analytics server 102.

The system 100 includes an analytics system 101 for hosting and implementing a clinical practice management system, a healthcare provider system 110 that includes a network infrastructure of computing hardware and software for providing care services by a care provider (e.g., physician, hospital, laboratory), patient devices 106a-106b (generally referred to as patient devices 106), and third-party servers 111a-111b (generally referred to as third-party servers 111). It is noted that patient information (e.g., personally identifiable or health information) may be protected from being passed to the third-party servers 111a-111b. The system 100 further includes any number of networks 105 for interconnecting the various components of the system 100. The analytics system 101 includes an analytics server 102 and analytics database 104. The healthcare provider system 110 includes various healthcare provider devices 103a-103e (generally referred to as provider devices 103), including a provider computing device 103a, provider server 103b, provider database 103c, and self-service kiosk device 103d. The patient devices 106 include a patient computing device 106a and patient mobile device 106b.

### Networks

The networks 105 of the system 100 include hardware and software components for interconnecting the various components of the system 100. The networks 105 may include any number of public networks and/or private networks to interconnect the various components of the system 100. Non-limiting examples of the networks 105 may include Local Area Network (LAN), Wireless Local Area Network (WLAN), Metropolitan Area Network (MAN), Wide Area Network (WAN), and the Internet. The communication over the network may be performed in accordance with various communication protocols, such as Transmission Control Protocol and Internet Protocol (TCP/IP), User Datagram Protocol (UDP), and IEEE communication protocols.

### Patient Devices

A patient device 106 includes a computing device that a patient operates to access and interact with the healthcare-related services of a care provider or analytics system 101 via the networks 105. For instance, the patient devices 106 may launch and execute an application or web browser to access a web-app or webpage hosted at the provider server 103b (or other device of the system 100), where the patient devices 106 transmits patient data as entered by the patient interacting with the web-app or webpage. The patient device 106 may be any computing device comprising hardware components (e.g., processor, non-transitory machine-readable storage media) and software components (e.g., software application(s), web browser), enabling the patient device 106 to perform the various operations and features described herein. Non-limiting examples of patient device 106 may include a patient computing device 106a and patient mobile device 106b (e.g., smartphone, wearable device), among others.

### Provider System

The provider system 110 represents a network infrastructure comprising physically and logically related software and electronic devices managed or operated for the healthcare provider. The provider system 110 comprises various hardware and software components that generates or otherwise obtains various types of patient-related data or metadata related to the patients, patient devices 106, or contact events for interactions between a patient and the care provider, including patient demographic data, patient-care records or history, and patient-care notes, among other potential types of data.

### Provider Computing Device

The provider computing device 103a includes any computing device allowing a healthcare provider user (e.g., physician, nurse, receptionist, researcher) to access, update, or otherwise interact with patient-related data. For instance, the provider computing device 103a includes software programming for a graphical user interface allowing the provider user to enter patient-related data and display various types of outputs from the analytics system 101, among other operations. The provider computing device 103a may be any computing device comprising hardware components (e.g., processor, non-transitory machine-readable storage media) and software components (e.g., software application(s), web browser), enabling the provider computing device 103a to perform the various operations and features described herein. Non-limiting examples of the provider computing device 103a may include a personal computer (e.g., workstation, laptop), mobile device, or tablet device, among other types of computing devices.

The care provider user may operate the provider computing device 103a to execute or access a practice management software programming (e.g., provider management app 120), which may be executed on the provider computing device 103a or on the analytics server 102. The provider management app 120 may instruct the analytics server 102 or provider computing device 103a to access historic patient data in patient data records stored in the provider database 103c or third-party server 111, such as Electronic Health Record (EHR) data records or credit-reporting service. In some cases, the provider computing device 103a accesses the provider management app 120 and executed at the analytics server 102, as cloud-based or remote-computing arrangement between the provider devices 103 and the analytics server 102. In some cases, the provider computing device 103a accesses the provider management app 120 and executed at the provider server 103b, as cloud-based or remote-computing arrangement between the provider devices 103 and the analytics server 103b. In some cases, the provider computing device 103a executes the provider management app 120 as locally installed and executed software program.

### Provider Server

The provider server 103b executes software programming for receiving patient-related from the provider devices 103 or other components of the system 100 and interacting with the patient devices 106 and analytics server 102 of the analytics system 101. The provider server 103b may be any computing device comprising hardware components (e.g., processor, non-transitory machine-readable storage media) and software components (e.g., software application(s), web browser), enabling the provider server 103b to perform the various operations and features described herein. Non-limiting examples of the provider server 103b may include a personal computer (e.g., workstation, laptop), mobile device, or tablet device, among other types of computing devices. For ease of description and understanding, the analytics server 102 performs the various operations of the machine-learning architectures, but embodiments are not so limited.

In some implementations, the provider server 103b may perform certain operations of the machine-learning architectures of the analytics server 102 described herein. As an example, the provider server 103b may execute the operations of the provider management app 120. In some implementations, the provider server 103b hosts a web portal or cloud-software programming that receives, stores, and updates patient-related data for the various devices of the system 100. For instance, the provider server 103b receives user inputs from patients or providers, as entered at a graphical user interface of a provider devices 103 or analytics server 102. The provider server 103b may update the patient data in the provider database 103c or send the patient data to a graphical user interface of a provider computing device 103a.

### Provider Database

The provider database 103c may be hosted on a computing device (e.g., server, desktop computer) of the healthcare provider system 110 comprising hardware and software components capable of performing the various processes and tasks described herein, such as non-transitory machine-readable storage media and database management software (DBMS). For instance, the provider database 103c may be hosted and executed by the provider server 103b or other provider device 103 of the healthcare provider system 110. The provider database 103c stores and maintains a comprehensive array of information for healthcare services. This includes patient profile records containing patient-related data, such as patient demographic data, which encapsulates personal details such as name, age, gender, and contact information, among other types of patient data. Additionally, the patient profile records of the provider database 103c may include care records (e.g., care treatments, payment histories, insurance provides), medical histories, information for past visits, diagnoses, treatments, procedures, and outcomes, among other types of patient data.

In some embodiments, the provider computing device 103a or the provider server 103b executes software programming for training or executing one or more machine-learning architectures, such as the machine-learning models described herein. At a training phase of the machine-learning architecture, the provider computing device 103a or the provider server 103b may reference patient data records of the provider database 103c as training data to train the machine-learning models of the machine-learning architecture. At a deployment phase (sometimes referred to “testing” or “inference time”), the provider computing device 103a or provider server 103b may reference or retrieve patient data records from the provider database 103c, and then feed the retrieved patient data to the trained machine-learning models of the machine-learning architecture of the provider management app 120.

### Self-Service Kiosk

A patient self-service kiosk device 103d can be used in a healthcare setting to streamline various administrative tasks and improve patient experience. The kiosk device 103d can be located at the care provider's office and configured to function as both a self-service terminal and a payment terminal. A patient can use the kiosk device 103d to sign in or check in for an appointment, fill out necessary forms, and make payments. The kiosk device 103d can be wirelessly connected to other provider devices 103 (e.g., provider computing device 103a, provider server 103b), which includes administrative software that can control or manage the kiosk device 103d. In at least some examples, the integration of these functions into a single device can reduce the need for multiple separate terminals, thereby optimizing space and resources in the medical office.

A patient can input or output various types of data information through various I/O components of the 103e, such as touchscreens, card readers, or barcode scanners, OCR scanners, among others. Non-limiting examples of patient inputs to the kiosk device 103d can include patient identification information, appointment details, insurance information, and payment details, among other types of patient-related data used by the software components of the analytics system 101 or other device of the system 100. Non-limiting examples of potential outputs from the kiosk device 103d can include printed receipts, appointment confirmations, and updated patient records. In at least some examples, the kiosk device 103d can also provide real-time feedback to patients, such as notifying the patients of any missing information or confirming the successful completion of their check-in process. In this example, the kiosk device 103d can enhance the efficiency of administrative tasks and improve the overall patient experience by providing a convenient and user-friendly interface for managing appointments and payments.

### Third-Party Servers

The third-party servers 111 include hardware components (e.g., processors, non-transitory machine-readable memory) and software components (e.g., software code of machine-learning architectures) for implementing the various processes described herein. The third-party servers 111 host various services that generate or store various types of data inputs for the machine-learning models of the analytics server 102 that, for example, generate estimates of the patient's financial responsibility and suggest the best possible payment plans and financing options or guarantees of payment, thus giving options at the point of service to reduce billing costs, waste, and lost patients. In some implementations, the third-party servers 111 execute processes or host data for relevant regulations or maintaining the integrity and confidentiality of patient information, where such data is ingested by the analytics server 102 for performing data security and compliance operations.

For ease of description and understanding, FIG. 1 depicts a first third-party server 111a and a second third-party server 111b, though embodiments may include any number of third-party servers 111 hosting or generating the various types of data used by the analytics server 102 to perform the processes and operations described herein. As an example, a first third-party server 111a executes processes for generate and hosting certain types of patient profile data. The first third-party server 111a may host or integrate EHR data to store and manage comprehensive patient profile data. This profile data may include detailed medical history, treatment records, and other relevant health information. The patient profile data of the first third-party server 111a may be ingested by an integration engine 128 of the analytics server 102 or provider server 103b to perform operations for personalized care and accurate billing.

As another example, a second third-party server 111b executes processes for hosting and generating patient financial information, such as financial data (e.g., credit report data) and/or insurance profile information. The third-party server 111b may contain patient data stored in financial and insurance databases to gather and store information about patient insurance policies, deductibles, co-payments, and co-insurances. The third-party server 111b may contain credit data of a credit reporting service that provides responses to requests messages generated and transmitted via a corresponding API by the integration engine 128 of the analytics server 102 or provider server 103b, such as messages from the integration engine 128 that request soft credit checks on patients. The responses may be received, processed, and stored by the integration engine 128, and ingested by the provider management app 120 of the analytics server 102 or provider server 103b for various operations.

### Analytics System

The analytics system 101 hosts a practice management system or RCM system for capturing and analyzing patient-related data and generating various types of outputs related to practice management. The analytics system 101 represents a network infrastructure comprising physically and logically related software and electronic devices managed or operated for a data analytics service provider. The analytics system 101 includes hardware and software components for obtaining the various types of data or information related to practice management and patients, including patient demographic data and patient-care records, among other potential types of patient-related data used for generating or updating patient profiles stored in a provider database 103c or analytics database 104. The devices of the network system infrastructure of the analytics system 101 are configured to provide the intended services for hosting the practice management system. The analytics system 101 hosts the practice management system as a remote or cloud-based service that performs the various functions, operations, and benefits on behalf of any number of healthcare provider systems 110 or similar healthcare related entities. For instance, physician offices, hospitals, medical researchers, or insurance providers may transmit or upload patient-related data or instructional commands (sometimes referred to as requests) to the practice management system of the analytics system 101 via the one or more networks 105, and the practice management system of the analytics system 101 may generate and return the outputs or instructional commands (sometimes referred to as responses) to the provider devices 103 via the one or more networks 105.

### Analytics Server

The analytics server 102 includes hardware components (e.g., processors, non-transitory machine-readable memory) and software components (e.g., software code of machine-learning architectures) for implementing the various processes described herein. The analytics server 102 may obtain (e.g., receive, retrieve) and process patient-related data received from the various types of data sources, such as the analytics database 104, provider devices 103, patient devices 106, and third-party servers 111, among others. Although FIG. 1 shows only a single analytics server 102, the analytics server 102 may include any number of computing devices. For instance, the analytics server 102 may host and include the analytics database 104 or may be in networked communication with another device of the analytics system 101 hosting the analytics database 104. In some cases, the computing devices of the analytics server 102 may provide all or portions of the processes and benefits of the analytics server 102. The analytics server 102 may comprise any number of computing devices operating in a distributed or cloud computing configuration and/or in a virtual machine configuration. Moreover, in some embodiments, functions of the analytics server 102 may be partly or entirely performed by other devices of the system 100, such as the provider devices 103 and/or the patient devices 106.

The analytics server 102 receives, retrieves, captures, or otherwise obtains various types of data related to a patient, where the patient-related data may be obtained as various types of data formats or modalities, such as text data or audio data, among others. The analytics server 102 executes software for ingesting and processing the patient data, including one or more machine-learning architectures having machine-learning models for ingesting and analyzing the patient-related data and producing various types of AI-generated patient data outputs. Non-limiting examples of the patient data sources or types of patient data may include patient-supplied data inputs from patient intake forms, patient data stored in EHRs, and voice recordings of patient-doctor interactions, among other relevant types of data and data sources.

The analytics server 102 includes software programming to execute, invoke, or otherwise perform various operations of one or more machine-learning architectures. These operations may include generating, training, or tuning layers of the machine-learning architectures. The analytics server 102 may train or tune executable layers of a machine-learning architecture into the analytics database 104. The analytics server 102 may further obtain and store, into the analytics database 104, a comprehensive dataset of patient data, which the analytics server 102 may reference in various operations, such as training data for training the machine-learning architectures of the analytics system 101.

The analytics server 102 executes the operations of a machine-learning architecture in several operational phases, including training operations for a training phase and deployment operations at a deployment phase (sometimes referred to as inference, test phase, or testing). The machine-learning architectures of the analytics server 102 ingest and process the various types of input data according to the operational phase. For example, the input data includes training data during a training phase for training parameters of the machine-learning architecture layers or includes input patient data during a deployment phase that the trained machine-learning architecture ingests and processes to generate the intended outputs or results.

The analytics server 102 may include software programming for performing operations for suggesting CPT and diagnosis codes to create a claim in real-time. These suggestions may be based on, for example, exams and narrative codes provided to the analytics server 102 by the provider (e.g., by the provider devices 103), patient (e.g., by the patient devices 106), and/or third-party (e.g., by the third-party servers 111). The suggested CPT and diagnostic codes are then used to automatically (or with approval) create a claim with the compliant codes.

The analytics server 102 includes software programming for performing operations for analyzing patient data and producing certain types of outputted information or graphical user interfaces for display at provider devices 103 or patient devices 106. For ease of description and understanding, the software operations are described and represented as software engines of the analytics server 102, though embodiments are not so limited. The software programming and functions may have any arrangement and need not be arranged as software engines described herein. Moreover, in some embodiments, the functions of certain software engines described herein may be performed by any number of additional or alternative computing devices of the system 100.

The analytics server 102 may include and execute a provider management app 120, form handling engine 122, and integration engine 128, among others. In some configurations, the respective software engines may execute or invoke software routines of the various machine-learning architectures described herein.

### Provider Management App

The provider management app 120 of the analytics server 102 is a software component of a clinical management software system or revenue cycle management system. The provider management app 120 includes software programming software routines, installed and executed at the analytics server 102 and/or other devices of the system 100 (e.g., provider computing device 103a, provider server 103b). The provider management app 120 is programmed to automate various processes or functions for clinical practice management, such as managing patient profiles or other financial management operations for a medical practice. This provider management app 120 includes software for machine-learning models of a machine-learning architecture trained to perform various operations of the analytics server 102. The provider management app 120 may ingest data from various data sources, which may be obtained via an integration engine 128.

The provider management app 120 may perform operations for predicting or generating suggested automated practice management operations (sometimes referred to as management actions), such as automated operations for addressing insurance or patient balances. For instance, the provider management app 120 may generate a suggested candidate management operation that would configure or instruct the computing devices of provider (e.g., analytics server 102) to automatically generate and send invoices to patients, as well as charge patient accounts based on outstanding balances.

The analytics server 102 may train a machine-learning model (e.g., an automation recommendation model) to generate suggested candidate automated practice management actions by using a large and diverse dataset as input. The training dataset for this automation recommendation model may include the various forms of clinical data, such as patient demographics, insurance information, healthcare service details, and historical billing information, among others. The analytics server 102 feeds the training data into the automation recommendation model and executes various training operations, such as back propagation, loss functions, and optimization algorithms, such as stochastic gradient descent, in order to adjust the parameters or weights of the automation recommendation model and minimize a level of error or loss between predicted outputs of the automation recommendation model and expected outputs (e.g., the actual training data indicated by training labels).

In an example, the automation recommendation model can be trained to identify or generate the suggested candidate automated actions, such as generating and sending invoices to patients, charging patient accounts based on outstanding balances, and addressing insurance claims. The automation recommendation model learns to recognize patterns and relationships within the training data, such as outstanding balances to make accurate predictions and suggestions. During the training process, a loss function or other layer of the machine-learning architecture updates the weights or parameters of the automation recommendation model through backpropagation, which involves calculating the gradient of the loss function with respect to the parameters of the automation recommendation model and using optimization algorithms to update the weights of various parameters of the automation recommendation model accordingly.

The provider management app 120 may perform operations for predicting or determining patient eligibility for healthcare services. For instance, the analytics server 102 or other device of the system 100 captures insurance information for the patient and determines whether the patient is eligible for a certain service (e.g., treatment, prescription, specialist). The analytics server 102 may obtain this insurance information for the patient from, for example, a patient device 106, provider computing device 103a, provider server 103b, kiosk device 103d, or third-party servers 111, which the analytics server 102 may store into the patient profile data in the provider database 103c or analytics database 104. In some cases, a patient mobile device 106b or kiosk device 103d may capture an image of the patient insurance card and send the image to the provider management app 120. The provider management app 120 may execute optical character recognition (OCR) operations to recognize characters of the insurance card, extract the patient insurance data, and store the patient insurance data into the patient profile data in the provider database 103c or analytics database 104. In some embodiments, the provider management app 120 creates a task for a staff member to manually check and confirm information extracted during OCR when the OCR scan has a low “assurance rating,” signifying a low confidence score in the accuracy of the OCR scan or extracted data.

The provider management app 120 may execute a machine-learning model (e.g., an eligibility model) using the insurance data of the patient profile or a live eligibility check through insurance data of patient profiles at the analytics database 104 or the third-party server 111b. This process involves querying the insurance database to verify the patient's coverage, including details such as deductible amounts, co-payments, co-insurance percentages, and out-of-pocket maximums. The provider management app 120 feeds this insurance-related data to the eligibility model to determine whether the patient is eligible for the requested healthcare service. In some cases, the provider management app 120 may further calculate the patient's financial responsibility as a patient's liability amount. The provider management app 120 system may continually update the trained machine-learning model to improve the accuracy of eligibility checks over time. In some embodiments, the provider management app 120 automatically queues adjustments in allowable amounts or Usual, Customary, and Reasonable (UCR) amounts (e.g., the amount actually expected to be paid on a given service from that specific payer) by scanning actual ERA payments, detecting differences and suggesting/automating with approval changes to the schedule of fees. The provider management app 120 can dynamically update the weights or parameters of the eligibility model and estimated patient liability outputs for the patient based on real-time changes in the patient's information or insurance coverage in the analytics database 104 or provider database 103c.

The analytics server 102 may train the eligibility model to determine patient eligibility by using a comprehensive dataset that includes various types of patient profile data relevant to patient eligibility checks. The analytics server 102 obtains training patient data for a training dataset from various data sources (e.g., third-party servers 111, provider database 103c, analytics database 104), such as patient demographics, insurance information, healthcare service details, and historical billing information, among others. The provider management app 120 may feed the training data into the eligibility model and executes various training operations, including back propagation, loss functions, and optimization algorithms, such as stochastic gradient descent, in order to adjust the parameters or weights of the eligibility model and minimize a level of error or loss between predicted outputs of the eligibility model and expected outputs (e.g., the actual training data indicated by training labels). In an example, the eligibility model can be trained to recognize patterns and relationships within the insurance information of the patient profile data that indicate patient eligibility for specific healthcare services. The training process involves backpropagation, where the parameters or weights of the eligibility model are updated based on the gradient of the loss function with respect to the eligibility model's parameters.

At deployment, the provider management app 120 obtains new eligibility requests indicating a patient via the user interface of the provider computing device 103a (or other device of the system 100). For instance, the eligibility request may include a patient identifier. The provider management app 120 retrieves patient profile data from the provider database 103c or other database using the patient identifier of the eligibility request. The provider management app 120 feeds the patient data to the trained eligibility model using new data inputs of the eligibility request, such as patient check-in information and insurance details, to determine patient eligibility in real-time. The provider management app 120 may generate and return an eligibility response containing an eligibility indicator for display at the user interface of the provider computing device 103a or other device of the system 100. Optionally, the eligibility model can dynamically update a predicted eligibility check output based on updates to the patient's information or insurance coverage in the patient profile data.

The provider management app 120 may perform operations for predicting or determining a patient liability amount for healthcare services. For instance, the analytics server 102 or other device of the system 100 captures various types of patient data, such as insurance information and payment information, and determines or generates the patient liability amount, which may include deploying a machine-learning model (e.g., liability model) on the various types of patient data. The analytics server 102 may obtain this insurance information for the patient from, for example, a patient device 106, provider computing device 103a, provider server 103b, kiosk device 103d, or third-party servers 111, which the analytics server 102 may store into the patient profile data in the provider database 103c or analytics database 104. In some cases, a patient mobile device 106b or kiosk device 103d may capture an image of the patient insurance card and send the image to the provider management app 120. The provider management app 120 may execute OCR operations to recognize characters of the insurance card, extract the patient insurance data, and store the patient insurance data into the patient profile data in the provider database 103c or analytics database 104. In some implementations, the provider management app 120 may generate an insurance profile as patient profile data using the patient's insurance information, which may include an insurance provider identifier, policy number, and coverage information, among other types of patient data.

The provider management app 120 may receive a request or instructions for determining the patient liability amount from the user interface of the provider computing device 103a or other device of the system 100, and the provider management app 120 may generate and return a response indicating the patient liability amount. In some cases, the provider management app 120 executes a patient liability model using the insurance profile data of the patient profile stored in one or more databases (e.g., provider database 103c, analytics database 104, third-party server 111b). The provider management app 120 may, for example, query an analytics database 104 or other database of the system 100 (e.g., provider database 103c, third-party servers 111) containing insurance data or insurance profile data to verify the patient's coverage for a particular healthcare service indicated by the request. The provider management app 120 feeds this insurance-related data to the liability model to determine the patient liability amount for a given healthcare service. The response may include details such as deductible amounts, co-payments, co-insurance percentages, and out-of-pocket maximums, which may be displayed at a user interface of the provider computing device 103a or other device of the system 100. In some embodiments, the system provider management app 120 automatically detects in-network and out-of-network payers based on provider settings. The provider management app 120 pulls relevant benefits and uses a different/custom patient portion calculation to calculate out-of-network costs and benefits. The provider management app 120 system may continually update the trained machine-learning model to improve the accuracy of predicted liability amounts over time. The provider management app 120 can dynamically update the weights or parameters of the liability model and estimated patient liability outputs for the patient based on real-time changes in the patient's information or insurance coverage in the analytics database 104 or provider database 103c.

The analytics server 102 may train the liability model to determine patient liability amounts by using a comprehensive dataset that includes various types of patient profile data, as financial responsibility for healthcare services. The analytics server 102 obtains training patient data for a training dataset from various data sources (e.g., third-party servers 111, provider database 103c, analytics database 104), such as patient demographics, insurance information, healthcare service details, and historical billing information, among others. The provider management app 120 may feed the training data into the liability model and executes various training operations, including back propagation, loss functions, and optimization algorithms, such as stochastic gradient descent, in order to adjust the parameters or weights of the liability model and minimize a level of error or loss between predicted outputs of the liability model and expected outputs (e.g., the actual training data indicated by training labels). In an example, the liability model is trained to recognize patterns and relationships within the data that indicate patient liability for healthcare services. The training process involves backpropagation, where the parameters or weights of the liability model are updated based on the gradient of the loss function with respect to the liability model's parameters.

At deployment, the provider management app 120 obtains new liability requests indicating a patient via the user interface of the provider computing device 103a (or other device of the system 100). For instance, the liability request may include a patient identifier, among other information (e.g., service identifier, expected service costs). The provider management app 120 retrieves patient profile data from the provider database 103c or other database using the patient identifier of the liability request. The provider management app 120 feeds the patient data to the trained liability model using new data inputs of the liability request, such as patient check-in information and insurance details, to determine a patient liability amount in real-time. The provider management app 120 may generate and return a liability response containing a liability indicator containing the liability amount for display at the user interface of the provider computing device 103a or other device of the system 100. Optionally, the liability model can dynamically update a predicted liability amount based on updates to the patient's information or insurance coverage in the patient profile data. The provider management app 120 dynamically updates balances as new services or quantities of existing services/products are administered, either through a front-office or clinical note interface.

The provider management app 120 may implement various machine-learning techniques or machine-learning models for a liability model to generate the estimated patient liability amounts. In some embodiments, the liability model implements a deep neural network (DNN) architecture, which can include deep learning models such as convolutional neural networks (CNNs) and recurrent neural networks (RNNs), among others. In an example, the liability model includes a CNN programmed and trained to process and analyze structured patient profile data, such as patient demographics and insurance information, by capturing spatial hierarchies in the patient data. The liability model may include a RNN programmed and trained handle sequential patient data, such as historical billing information and payment patterns, by capturing temporal dependencies.

In some embodiments, the analytics server 102 trains the liability model using ensemble learning techniques, such as random forests and gradient boosting machines (GBMs), among others. In an example, random forests can combine multiple decision trees to improve the liability model accuracy and robustness by reducing overfitting. In this example, a GBM can iteratively build and combine weak learners to create a strong predictive model, enhancing the model's performance in estimating patient liability. In some embodiments, the liability model can include layers for support vector machines (SVMs) for classification operations, such as determining patient eligibility based on insurance information. In an example, SVMs can find the optimal hyperplane that separates different classes in the data, ensuring accurate classification of patient eligibility. In other examples, the liability model can incorporate natural language processing (NLP) techniques to analyze unstructured patient data obtained from the various data sources, such as financial or insurance information from third-party servers 111 or and patient responses entered at a user interface of the patient devices 106. In an example, NLP techniques like word embeddings and transformers can be used to extract meaningful features from text data, which the liability model references to estimate patient liability based on comprehensive patient information.

### Form Handling Engine for Custom Forms and Templates

The form handling engine 122 provides adaptable templates that can capture nuances of individual patient encounters. The flexibility of the form handling engine 122 provides flexible data gather and comprehensive clinical documentation. The same or different AI/ML model trained for automatic narrative generation (e.g., the LLM of the provider management app 120) can be trained to convert and ingest customized patient intake forms to generate simple narratives for the patient intakes notes.

In some implementations, the form handling engine 122 includes software routines allowing a healthcare provider to generate and map customized intake-form templates via a graphical user interface to certain aspects of healthcare treatments, payments, and appointments, and other aspects of practice management, thereby configuring the machine-learning architecture of the form handling engine 122 to generate narratives that are tailored to the specific patient's healthcare services (e.g., appointments, treatments) and liabilities.

In some embodiments, the form handling engine 122 includes a form configuration portal hosted on the analytics server 102. The form configuration portal includes a graphical user interface and software programming allowing healthcare providers to create, customize, and manage customized patient intake forms, such as a patient insurance form or patient payment form. The portal provides a graphical user interface that allows the provider to design and personalize intake form templates. Utilizing drag-and-drop functionalities, providers can add, remove, and rearrange form fields to suit specific requirements or provider specialties. The provider may choose from a library of pre-existing templates or create new forms hosted in the analytics database 104. A form template can be customized to include specific data fields relevant to the patient's visit, such as demographic information, medical history, payment information, the provider's specialty, or dynamic, interactive images, such as a 2D body chart or 3D body chart with labeling/drawing. In this way, the portal of the form handling engine 122 allows the provider to tailor the templates to the needs of various clinical scenarios.

In some cases, the form handling engine 122 integrates real-time data from EHRs and other clinical data sources into a patient intake form. As an example, as the patient enters a patient intake form, the form handling engine 122 may automatically pull relevant information from the various data sources to complete the data fields of the patient intake fields, minimizing manual data entry and reducing the risk of errors.

As with the provider management app 120, a machine-learning model of the form handling engine 122 may include a transformer-based model (e.g., LLM, BERT, GPT), for handling complex language tasks and generating coherent text. In an example, the machine-learning model can be implemented by analytics server 102 to handle customized forms by analyzing the input data and generating appropriate outputs. The inputs to the machine-learning model of the form handling engine 122 can include various types of structured and unstructured data, such as data fields of patient intake forms, EHRs, insurance information, and other relevant patient-related data. The provider management app 120 may create custom reports in various formats/graphs using data available in the system 100, such as data from the revenue cycle management and business analytics (e.g., “Profit and Loss”, comparative analysis of difference resources, etc.). In some examples, the outputs from the machine-learning model (e.g., form handling model) of the form handling engine 122 can be structured data entries, generated narratives, or other forms of processed information that can be integrated into other data records of the patient or other software components of the analytics system 101. The provider management app 120 may use custom mapping of unstructured data based on keywords to convert the unstructured data to structured data. For example, a suggested CPT code is mapped to a coding template to transfer automatically to the claim. In clinical settings, keywords such as “subjective,” “HPI,” “ROS,” whether spelled out or abbreviated, automatically map LLM outputs to the subjective clinical section, subject to the provider's approval and sign-off.

The training process for the machine-learning model of the form handling model involves providing a large and diverse dataset as input, which includes various forms of clinical data. In this example, the output of the training process is a trained form handling model that can accurately process and generate customized forms. In some examples, the weights of the form handling model are updated during backpropagation by comparing the predictions generated by the form handling against the actual data (e.g., training data, expected data, real patient data) and adjusting the parameters to minimize the error. In at least some examples, this training process involves calculating the gradient of the loss function with respect to the parameters or weights of the form handling model and using optimization algorithms, such as stochastic gradient descent, to update the weights of the form handling model accordingly. In this example, the trained form handling model can then be deployed on the analytics server 102 to handle customized forms based on new clinical data inputs.

At deployment, as healthcare providers fill out different inputs in the custom templates, the form handling engine 122 can invoke and execute the form handling model having an LLM or similar form of machine-learning model to generate narratives in real-time, similar to how a large language model (LLM) would generate text based on the provided information. In this example, the form handling engine 122 can be configured to enhance these simple narratives using the LLM of the form handling model, automatically transforming the narratives into more detailed and coherent narratives. In some examples, the form handling engine 122 can provide real-time feedback, such as notifying providers of any missing information or confirming the successful completion of the documentation process. This approach improves the overall efficiency and accuracy of clinical documentation by providing a convenient and user-friendly interface for managing customized templates. The form handling engine 122 may also convert detailed narratives into “clinical summaries” (e.g., short form notes with codes) using the information of the form and/or other types of inputs, such as care-related inputs entered by a doctor during a visit. This may be useful in clinical settings in which clinical summaries are more common, such as worker's compensation or other legal situations, as well as physical medicine. The form handling engine 122 formats the data into simple narratives, such as in bulleted format with preselected or AI-suggested codes, identified or generated by the form handling model. In some embodiments, a discharge summary and patient education materials are automatically generated using the data from the note, intake, patient chart or other encounter.

In some cases, the LLM of the form handling model is instructed through the LLM prompts to avoid generating information that is not present in the patient-related data, thereby avoiding hallucinations. This is crucial in the medical field, as accuracy and reliability of the generated text are critical. In such cases, the LLM prompts explicitly instruct the LLM to use only the provided patient data and avoid assumptions or adding extraneous information. In some cases, the LLM prompts enable or tune the LLM to incorporate medical terminology and context. For example, the LLM uses the LLM prompts to understand the context of the patient's medical history, current symptoms, and treatment plans, among other types of data inputs. In some cases, the LLM processes the input data and generates a clinical note, that includes a suggested CPT code (e.g., an Evaluation and Management (E&M) code) based on the recognized medical procedures and diagnoses (e.g., ICD codes, such as ICD-10 codes) in the patient data inputs. For instance, the LLM may be trained to recognize patterns in the training patient clinical data to suggest appropriate CPT codes based on the content of the clinical notes during the deployment phase. In some embodiments, the LLM is configured to return an error and/or note in the event that one or more of the previous outputs of the LLM does not meet standards for a billing level and then provides a suggestion on whether to adjust the error-identified format/information (e.g., upping a code of the note and/or adding additional information) such that the output conforms to the various compliance requirements. This allows for an efficient flow of information between clinical AI models and RCM systems within a healthcare system to expedite claim creation/payment with reduced claim rejections.

For instance, when a patient has completed an intake form, the form handling engine 122 invokes the LLM of the form handling model or the provider management app 120 to produce detailed narratives based on the provided information in the patient intake form and other data sources. In some cases, the narratives generated for the form intake may be generated as structured data for inclusion into a portion of a patient profile used by the various machine-learning models or operations of the provider management app 120. In some cases, the form handling engine 122 generates notification outputs for display at a user interface of the patient devices 106 the provider devices 103, based on data entered into the data fields of the intake form and other patient-related data from the other data sources. For example, the form handling engine 122 can detect missing or incomplete fields of the patient intake form and notify the provider of any missing information or confirm the successful completion of the form.

As an example, the provider may access the portal to configure an browser-based form interface or similar interface (e.g., PDF form file), such as a vaccine form. To configure a vaccine intake form, the prover operates the provider computing device 103a to access the form configuration portal of the form handling engine 122 on the analytics server 102. The provider selects a pre-existing vaccine template or creates a new template from scratch. The provider may interact with configuration features of the portals, such as a drag-and-drop functionality to add relevant fields, such as patient name, date of birth, vaccine type, dosage, and administration site. In some embodiments, demographic information (e.g., name, date, etc.) is automatically pulled from the patient chart. The form handling engine 122 instructs the analytics server 102 access various other data sources, such that form handling engine 122 may integrate real-time data from the other data sources, such as EHRs, to auto-populate the types of fields. In some implementations, the form handling engine 122 may incorporate patient-related into the data fields with existing patient information for a particular patient. The analytics server 102 may store the intake template form into the analytics database 104 or provider database 103c for future use or generate the intake form for immediate patient documentation. At the time of patient intake, the patient operates the patient devices 106 or kiosk device 103d to access one or more patient intake forms, which are presented at the user interface of the patient device 106 or kiosk device 103d, allowing the patient to enter information in the data fields via the graphical user interface. Upon completion, the machine-learning model of the form handling engine 122 may generate a detailed narrative of the patient's intake forms using the provider management app 120.

### Integration Engine

The integration software engine 128 may capture and integrate various types of patient data and practice data, such as patient profile data and financial management data, among other types of data, from any number of data sources. The integration engine 128 may obtain (e.g., receives or retrieves) patient medical history data records, such as EHRs stored in the provider server 103b or third-party servers 111, including treatment records and diagnostic information. In The integration engine 128 may obtain (e.g., receive, retrieve) patient profile data, which may include demographic information, such as age, gender, and contact details, among others. The integration engine 128 may also obtain (e.g., receive or retrieve) various patient-related financial data, such as financial information and insurance information (e.g., policy details, deductibles, co-payments, co-insurances).

The integration engine 128 may obtain and store the various types of data into a provider database 103c and/or analytics database 104, such that the various types of data obtained by the integration engine 128 is accessible to the software components of the analytics system 101 or healthcare provider system 110. As an example, the integration engine 128 may obtain the various types of patient and practice data from the provider server 103b or third-party servers 111, such that the integration engine 128 may access data indicative of a patient financial responsibilities, such as patient insurance information, patient financial data (e.g., credit scores, payment histories) from the provider server 103b or third-party servers 111, such that integration engine 128 may access data for determining personalized payment plans and financing options.

The integration engine 128 includes application programming interfaces (APIs) to obtain the various types of data from external systems. The operations of the integration engine 128 harmonizes various data sources. This integration engine 128 may reduce redundant data entries and minimize discrepancies within patient records. In some examples, the integration engine 128 can aggregate data from multiple sources within the system 100, which may include the insurance information or financial data from the third-party servers 111 or patient-related data stored in a provider server 103b, such as patient appointments, medication lists, and treatment plans, among other types of data.

The integration engine 128 may obtain in response to real-time requests received via the provider computing device 103a or patient devices 106, or at different intervals, such as scheduled intervals or periodic intervals. As an example, the integration engine 128 can also pull insurance information from insurance databases of the third-party servers 111 at regular intervals to reflect changes in policy details for patients. In some examples, the integration engine 128 can perform real-time requests to credit reporting services to obtain the latest credit scores and financial data. The integration engine 128 can use batch processing to update demographic information from hospital registration systems periodically.

The data sources may include the EHR data stored in the third-party servers 111, which store comprehensive patient information and medical history data. In this example, the integration engine 128 can also retrieve patent data from insurance databases hosted in third-party servers 111, which contain detailed information about insurance plans, coverage, and patient eligibility. In some examples, the integration engine 128 can access billing systems to obtain historical billing information and payment patterns. In at least some examples, the integration engine 128 can also pull data from real-time sources, such as check-in form data entered via user interfaces of patient check-in kiosk devices 103d or mobile applications of patient devices 106, where patients can enter information and insurance details. In some implementations, the integration engine 128 can perform periodic data pulls at scheduled intervals to update one or more machine-learning models with the latest patient information. The integration engine 128 may also generate or handle real-time requests for data retrieval, such as when a patient checks in for an appointment and provides insurance information.

In some implementations, the integration engine 128 stores the patient data for other software components of the analytics server 102. For instance, the integration engine 128 may periodically retrieve patient financial data from a third-party server 111 or receive patient financial data from patient devices 106, which the integration engine 128 may ingest, format, and store into the provider database 103c or analytics database 104. The analytics server 102 or provider server 103b may detect, retrieve, and feed this updated into one or more machine-learning models of the provider management app 120, such that analytics server 102 or provider server 103b may dynamically update the estimated patient liability based on real-time changes in the patient's information or insurance coverage (or contracted allowable amounts with specific payers), or tune the machine-learning models. In at least some examples, the integration engine 128 stores the information and the provider management app 120 generates periodic or real-time updates to a dashboard user interface accessible to the healthcare providers and patients via the provider computing device 103a or patient devices 106, allowing the providers or patients to review and understand, for example, the estimated costs or other data ingested by the integration engine 128.

The provider management app 120 may access and invoke an integration engine 128 to perform certain operations for analyzing third-party patient data and generating proposed payment options using the third-party patient data, among other operations using the third-party data, such as a credit report data stored at a third-party server 111b.

The integration engine 128 or other software component of the analytics server 102 may perform various preprocessing operations on the patient-related data. The preprocessing operations may clean and normalize the various data inputs. As an example, missing values are imputed, and categorical variables are encoded into numerical representations. In this example, the data is then scaled to ensure that all features are on a similar scale, which helps improve the model's performance. In some examples, the data is also split into training and validation sets to evaluate the model's performance and prevent overfitting. In some cases, the integration engine 128 or other software component of the analytics server 102 may execute operations to extract one or more features or feature vectors from the various types of data obtained by the analytics server 102.

### Analytics Database 104

The analytics database 104 stores various types of data or information used for training, tuning, or deploying the various executable engines or machine-learning architectures of the analytics server 102. The analytics database 104 may be hosted on any computing device (e.g., server, desktop computer) comprising hardware and software components capable of performing the various processes and tasks described herein, such as non-transitory machine-readable storage media and database management software (DBMS).

The analytics database 104 or other databases of the system 100 (e.g., provider database 103c, third-party server 111a) may store one or more corpora of training data (e.g., training patient data, training metadata), accessible to the analytics server 102 via the one or more networks 105. In some embodiments, the analytics server 102 may employ supervised training to train the one or more machine-learning models of the machine-learning architecture(s) discussed herein. In such embodiments, the analytics database 104 and/or other databases contain training labels associated with the training data. For a particular machine-learning model, the labels indicate, for example, the expected data or expected outputs when the machine-learning model is applied to the training data. The analytics server 102 may also query an external database (e.g., provider database 103c, third-party server 111b) to access additional or alternative training data.

### Example Processes

### AI/ML Recommending Clinical Management Operations

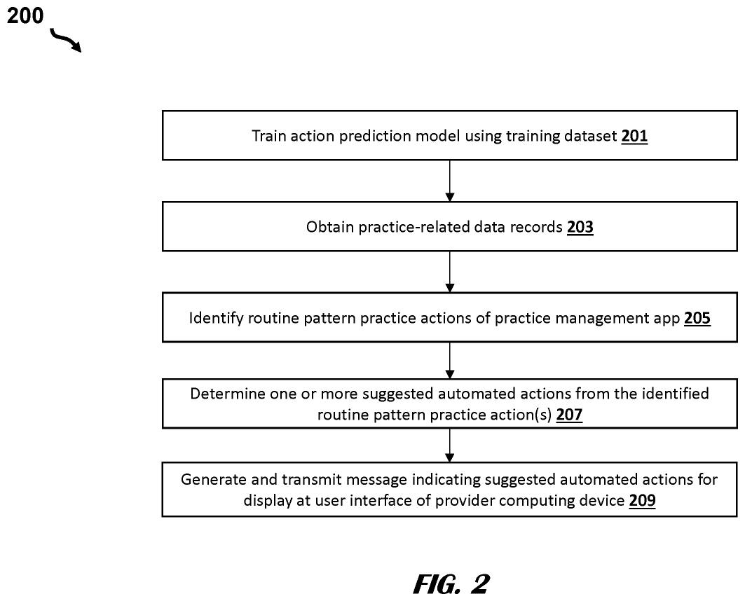

FIG. 2 shows a flowchart of operations of a computer-implemented method 200 for identifying and suggesting automated operations (or actions), such as device-executed software operations for addressing insurance and patient balances (e.g., sending invoices, charging patient accounts), among other types of automated operations. A computing device (e.g., analytics server 102, provider server 103b), which may be part of a management system, executes software programming (e.g., provider management app 120) of one or more machine-learning models (e.g., an automation recommendation model) to ingest various types of practice data (e.g., scheduling data, invoice cycle data, payment cycle data, insurance data, and/or patient profile data), identify one or more frequent pattern operations performed by a management application, and generate one or more suggested candidate operations to be automated by devices or software components within the management system. In some embodiments, the computing device further generates and transmits messages for display at a management computing device (e.g., provider computing device 103a) indicating the suggested candidate operations and associated scores. For ease of description and understanding, the operations of the computer-implemented method 200 are described as being performed by a computer, though embodiments may be implemented using any type of computing device and/or may be implemented using multiple computing devices.

In operation 201, the computer trains one or more machine-learning models as an automation recommendation model using a training dataset. The computer may train the automation recommendation model using historical data related to patient profiles, payments, insurance claims, and invoicing history, among other types of data indicative of patterns of various practice management operations.

In an example, the automation recommendation model can be trained to identify pattern operations (sometimes referred to as routine or regular operations or actions) performed by a healthcare practice system by capturing and analyzing historical data related to the various operations performed using a provider management app of a practice management system. The historical data or training data can include patient check-ins, appointment scheduling, billing processes, insurance claim submissions, and communication logs. The automation recommendation model extracts a feature vector from this data, representing various attributes of the pattern operations, such as the frequency of appointments, the average time taken for billing, the success rate of insurance claims, and common patient inquiries. In some implementations, the computer executes natural language processing (NLP) programming or techniques to analyze communication logs or other textual data to identify or extract patient profile data or other types of data from the historical data or training data.

The automation recommendation model may include various types of machine-learning models or machine-learning techniques, such as decision trees, random forests, support vector machines, and neural networks, among others. For example, a decision tree model can be used to classify insurance claims based on a claim approval score indicating a likelihood of approval or denial. A neural network model of the automation recommendation automation recommendation may be programmed and trained to identify and analyze payment patterns to predict overdue payments and suggest an automated operation for sending reminders to patients by transmitting reminder notifications to patient devices via one or more communication channels (e.g., email, SMS text message, phone call). The training data may be referenced by the automation recommendation model to identify patterns and relationships that the automation recommendation model learns to recognize, such as billing cycles for patients, payment cycles for patients, and insurance coverage requests, among others. The training process involves using a loss function to update the automation recommendation model weights or parameters through backpropagation, which calculates the gradient of the loss function with respect to the weight or parameters of the automation recommendation model and uses optimization and backpropagation to adjust the parameters or weights of the automation recommendation model.

In an example, the automation recommendation model is trained to identify pattern operations performed by computing devices of a healthcare practice system (e.g., analytics server 102, practice server 103b) by first collecting and analyzing historical data or other types of training data related to the operations performed using the provider management app. The training data can include patient check-ins, appointment scheduling, billing processes, insurance claim submissions, and communication logs. The automation recommendation model extracts a training feature vector from this training data, representing various attributes of the routine pattern operations, such as the frequency of appointments, the average time taken for billing, the success rate of insurance claims, and common patient inquiries.

In some cases, the training process involves using supervised learning techniques where the automation recommendation model learns to recognize patterns and relationships within the training data that indicate routine, frequent pattern operations. For instance, a neural network model of the automation recommendation model can be used to process the training feature vectors through a series of layers of the automation recommendation model. The input layer receives the training feature vector, and each subsequent layer applies a set of parameters or weights and activation functions to transform the training data or other input data. The hidden layers identify complex patterns and relationships within the training data, allowing the automation recommendation model to learn from historical examples of pattern operations and corresponding outcomes.

The automation recommendation model may include a loss function to update or adjust weights or parameters through backpropagation. This involves calculating a gradient of the loss function with respect to the automation recommendation model parameters and using optimization algorithms, such as gradient descent, to update the parameters or weights of the automation recommendation model accordingly. The automation recommendation model is trained by minimizing the loss function, which measures the difference between the predicted pattern operations or outcomes of pattern operations and actual pattern operations or outcomes of pattern operations.

In another example, the automation recommendation model can use a decision tree to identify routine or frequent pattern operations. The decision tree splits the feature vector into branches based on specific criteria, such as the type of pattern operation or the time taken to complete the pattern operation. Each branch represents a decision point, and the automation recommendation model follows the branches to reach a final decision node that indicates the pattern operation. The decision tree is trained using historical data, where each branch and decision point is optimized to accurately classify pattern operations based on attributes or features of the various operations that may be performed by computing devices or software components of the provider system.

Using the training data, the automation recommendation model is further trained to determine a suggested candidate automated operation from the identified pattern operations. In this way, the automation recommendation model may identify frequent or routine pattern operations and then identify one or more suggested automated operations corresponding to one or more of the identified pattern operations For example, if the automation recommendation model recognizes that appointment scheduling frequently involves checking a physician's availability and sending reminders to patients, the automation recommendation model can suggest automating one or more of these pattern operations. The automation recommendation model generates a suggested automated operation, such as automatically scheduling appointments based on provider availability and sending reminders to patient devices via email or SMS.

To train the automation recommendation model to determine whether a pattern operation can be automated, the computer applies the automation recommendation model against training data for historical provider management operation data. This provider training data includes various provider-management operations, such as computer or software operations executed when scheduling appointments, updating patient profiles, processing payments, and managing insurance claims. The automation recommendation model uses various machine-learning techniques to identify suggested automated operations. For instance, a decision tree model can classify operations based on potential for automation by analyzing attributes, such as time taken, error rates, and workflow impact. A neural network model of the automation recommendation model can analyze complex patterns in the data associated with a pattern operation, as a candidate automated operation, to predict the efficiency and effectiveness of automating the pattern operation with higher accuracy. The automation recommendation model processes the input data, extracts relevant features, and generates a candidate operation score indicating a likelihood that automating the candidate operation corresponding to the pattern operation will be beneficial.

The automation recommendation model compares the candidate operation score (corresponding to a particular pattern operation) against an automation threshold. In some embodiments, the automation threshold is a predefined threshold score (e.g., a numeric value, a percentile, or a probability threshold) stored in memory and configurable per provider, per practice, per payer, per operation type, or per workflow. In some embodiments, the automation threshold is determined or tuned based on historical outcomes (e.g., reductions in errors, reduced days-in-accounts-receivable, increased collection rate, reduced staff time, or increased patient satisfaction) for prior automated operations and/or based on a target false-positive rate for automation recommendations. If the automation recommendation model determines that the candidate operation score satisfies the automation threshold, then the automation recommendation model identifies the corresponding pattern operation as a candidate automated operation and the management application generates an instruction to execute the candidate automated operation (e.g., automatically scheduling appointments based on provider availability and transmitting reminder notifications to patient devices via email or SMS).

In operation 203, the computer obtains (e.g., retrieves, receives) practice-related data records, which may include the types of data of the training dataset. The computer may continually obtain the types of data at a preconfigured interval or in response to updates at one or more databases (e.g., provider database 103c, third-party servers 111) The computer captures various types of data to identify any number of pattern operations, identify one or more candidate automated operations in the pattern operations, and determine suggested automated operations in the candidate automated operations. This data can include patient demographics, medical history, insurance details, payment records, appointment schedules, and communication logs. The computer can also capture real-time data such as patient check-ins, insurance verifications, and payment transactions. By analyzing this data, the automation recommendation model can identify patterns and trends to detect one or more pattern operations.

The computer retrieves data from various types of data sources at different intervals or in response to real-time requests. Data sources can include electronic health records (EHR) systems, billing systems, insurance databases, patient portals, and third-party services. The system can pull data at regular intervals, such as daily or weekly, to ensure that the information of the data is up-to-date. In some examples, the computer can also retrieve data in real-time, such as in response to a trigger indication or instruction when a patient checks in or when an insurance claim is submitted, to generate and provide immediate, responsive, real-time suggestions for suggested automated operations.

In operation 205, the computer identifies one or more pattern management operations (sometimes referred to as frequent routine practice management actions) executed by a practice management app of a practice management system. The automation recommendation model identifies a pattern operation (or frequent routine operation or action) performed by a healthcare practice system by obtaining and analyzing historical data related to provider-management actions performed by a provider management application. This practice operation data (or practice action data) can include provider management operations, such as scheduling appointments, updating patient profiles, processing payments, finalizing or transmitting invoices, and managing insurance claims, among others. The automation recommendation model extracts a feature vector from this practice operation data, representing various attributes of the provider-management operations, such as the frequency of appointments, the average time taken for billing, the success rate of insurance claims, and common patient inquiries. The input layer of the neural network receives the feature vector, and each subsequent layer applies a set of weights and activation functions to transform the input practice operation data.

For example, for a provider management operation of scheduling appointments, the automation recommendation model obtains (e.g., receives, retrieves, extracts, generates) operation data on the number of appointments scheduled per day, the time slots most frequently booked, the average duration of appointments, and the rate of appointment cancellations or rescheduling, among others. The automation recommendation model transforms the practice management operation data into a feature vector, which includes attributes such as the daily appointment count, preferred time slots, average appointment duration, and cancellation rate.

In some implementations, the computer stores and maintains a set of expected routine features representing one or more expected pattern operations. Non-limiting examples of the set of expected routine features may include one or more template feature vectors associated with known operation types (e.g., “send invoice,” “send appointment reminder,” “verify eligibility”), (ii) a learned centroid or embedding representing a cluster of historical feature vectors for a given operation type, and/or rules or constraints defining typical ranges of feature values for an operation type. In some embodiments, the computer generates a pattern operation score (also referred to as a routine operation score) that represents a likelihood that the current operation is a pattern operation by comparing the current feature vector to the set of expected routine features (e.g., by computing a similarity metric, distance metric, or classification probability).

In addition or as an alternative to the pattern operation score, the computer determines whether the operation occurs with sufficient regularity by determining whether a number of instances of the operation satisfies a regularity threshold number of instances. In some embodiments, the regularity threshold number of instances is evaluated over a time window rate or metric (e.g., N occurrences within the last X days/weeks/months) and/or according to a preconfigured interval (e.g., occurring approximately weekly, daily, or per-visit), and may be configurable based on an operation type. When the pattern operation score satisfies a pattern threshold and the number of instances satisfies the regularity threshold number of instances, the computer identifies the operation as a pattern operation eligible for further automation scoring.

In operation 207, the computer determines one or more suggested or candidate automated operations from the one or more routine practice pattern operations as previously identified. For each identified routine pattern operation, the automation recommendation model determines a suggested automated operation using the extracted feature vectors representing the identified routine pattern operation. Once a routine pattern operation is identified, the automation recommendation model determines whether the pattern operation can be automated by evaluating the efficiency and effectiveness of automating the pattern operation. The automation recommendation model is trained to considers factors such as the time saved, the reduction in errors, and the overall improvement in workflow. If the automation recommendation model determines that automating the action would be beneficial, then the automation recommendation model generates a suggested automated operation or otherwise identifies the pattern as a suggested automated operation. As an example, if the automation recommendation model recognizes that appointment scheduling frequently involves checking provider availability and sending reminders to patients, then the automation recommendation model can suggest automating these provider operations of the provider management app. In this example, the automation recommendation model generates a suggested automated operation, such as automatically scheduling appointments based on provider availability and sending reminders to patients via email or SMS. In another example, the automation recommendation model can identify routine billing processes that involve generating invoices and tracking payments. By analyzing the feature vector, the automation recommendation model can suggest automating the generation of invoices and sending payment reminders to patients with outstanding balances.

This provider operation data includes the various provider-management operations, such as scheduling appointments, updating patient profiles, processing payments, and managing insurance claims. The automation recommendation model uses various machine-learning techniques to identify suggested automated operations. For instance, a decision tree model of the automation recommendation model can classify actions based on the potential for automation by analyzing attributes, such as time taken, error rates, and workflow impact. A neural network model of the automated recommendation model can analyze complex patterns in the data to predict the efficiency and effectiveness of automating the candidate pattern operation with higher accuracy. The automation recommendation model processes the input data, extracts relevant features, and generates one or more scores, such as a candidate operation score indicating the likelihood of automation being beneficial. The automation recommendation model generates the candidate operation score, which is a numerical value representing the likelihood that automating the candidate operation will result in significant improvements. The automation recommendation model determines this candidate operation score based on the evaluation of the feature vector representing the features extracted for the identified frequent or routine pattern operation. For instance, the automation recommendation model might assign higher weights to operations that save more time or reduce errors more effectively. The candidate operation score is generated using machine-learning techniques, such as logistic regression or neural networks, which process the input data and produce a probability score indicating the potential benefits of automation.

The automation recommendation model compares this suggested automated score against a suggested candidate automation threshold score. If the automation recommendation model determines that automating the candidate pattern operation would be beneficial, the automation recommendation model identifies the routine pattern operation as a suggested automated operation. For example, if the automation recommendation model determines that automating appointment scheduling would save time, reduce errors, and improve workflow, then automation recommendation model determines that automatically scheduling appointments based on provider availability and sending reminders to patients via email or SMS should be a suggested automated operation.

For instance, the automation recommendation model is trained to determine whether a routine pattern operation should be automated by evaluating the potential benefits and effectiveness of automation. The computer captures and analyzes the training or ongoing historical provider management operation data related to provider-management operations, such as scheduling appointments, updating patient profiles, processing payments, and managing insurance claims. This data is extracted or transformed into feature vectors, which are numerical representations of the attributes of these operations. The model processes these feature vectors through the various layers, capturing complex patterns and relationships within the data.

To calculate the candidate operation score, the automation recommendation model evaluates several factors represented as features or feature vectors; for instance, a set of improvement features representing improved management operations. In some embodiments, the set of improvement features includes, for a given operation type or candidate automated operation, one or more features representing expected improvements (or predicted improvements) in one or more performance metrics relative to a baseline (e.g., a non-automated execution). Non-limiting examples of improvement features include expected staff time savings, reduced error rate, improved completion rate, reduced days-in-accounts-receivable, reduced claim rejections, increased collection rate, reduced patient call volume, and/or improved patient satisfaction. In some embodiments, improvement features are computed from historical outcomes of prior operations (e.g., historical logs indicating time-to-complete, corrections, rework, or downstream denials), from simulation, and/or as outputs of one or more trained prediction models.

In some cases, the automation recommendation model estimates an amount of time that could be saved by automating a pattern operation. For example, if the computer determines that scheduling an event or appointment according to user inputs takes an average of 5 minutes per appointment and the practice schedules 50 appointments per day, then the computer may determine automating that the scheduling process could save approximately 250 minutes per day. In some cases, the automation recommendation model assesses the potential reduction in errors, such as double bookings or incorrect time slots, by comparing a baseline error rate of manual scheduling to an expected error rate of an automated process. In some cases, the automation recommendation model considers overall workflow improvement, such as increased efficiency and better utilization of provider availability, by evaluating how automation could streamline the scheduling process and reduce administrative burden. The candidate operation score may be generated based on, for example, the feature vector representing the pattern operation and the set of improvement features representing improved management operations.

The automation recommendation model combines these factors to generate a probability score indicating the potential benefits of automation. For instance, the model might assign higher weights to operations that save more time or reduce errors more effectively. The score is calculated using machine-learning techniques, such as logistic regression or neural networks, which process the input data and produce a numerical value representing the likelihood that automating the operation will result in significant improvements.

If the suggested automated operation score exceeds a predefined threshold, the model generates a suggested automated operation, such as automatically scheduling appointments based on provider availability and sending reminders to patients via email or SMS. This score helps healthcare providers make informed decisions about which routine pattern operations to automate, enhancing the efficiency and effectiveness of the practice system.

In operation 209, the generates and transmits a message indicating the one or more suggested automated operations for display at the user interface of a provider computing device. Optionally, the computer continuously learns and improves the automation recommendation model over time by analyzing new practice-related data and incorporating feedback from the healthcare provider users via one or more provider computing devices. The computer may, for example, implement backpropagation and reinforcement learning techniques to optimize weights or parameters of the automation recommendation model to improve the suggested automated operations based on the outcomes of previous suggested automated operations.

### Patient Eligibility Check

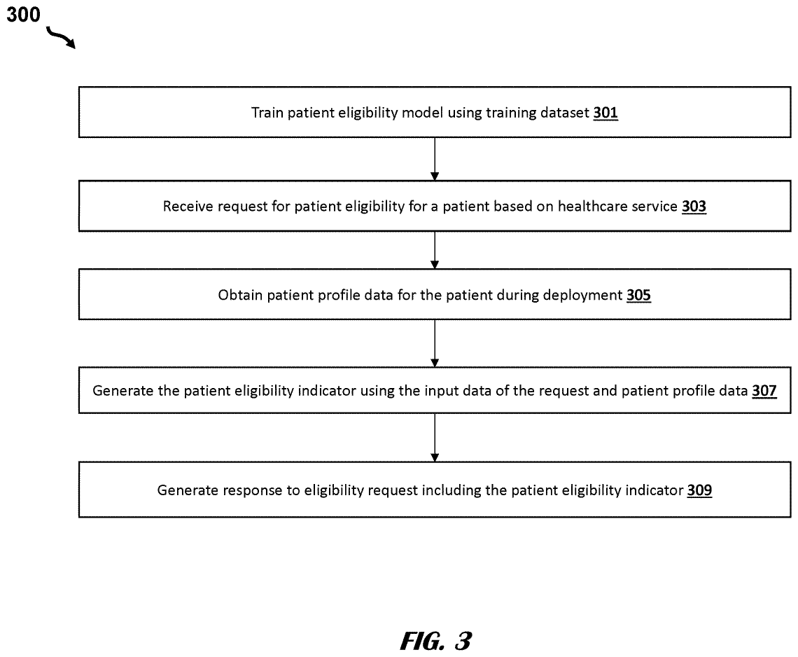

FIG. 3 shows a flowchart of operations of a computer-implemented method 300 for determining or predicting patient eligibility for a healthcare service. A computing device (e.g., analytics server 102, provider server 103b) executes software programming (e.g., provider management app 120) of a machine-learning model (e.g., patient estimate model) for ingesting various types of patient-related data from various data sources, determining healthcare services available to the patient, and generating or predicting the patient eligibility. For ease of description and understanding, the operations of the computer-implemented method 300 are described as being performed by a computer, though embodiments may be implemented using any type of computing device and/or may be implemented using multiple computing devices.

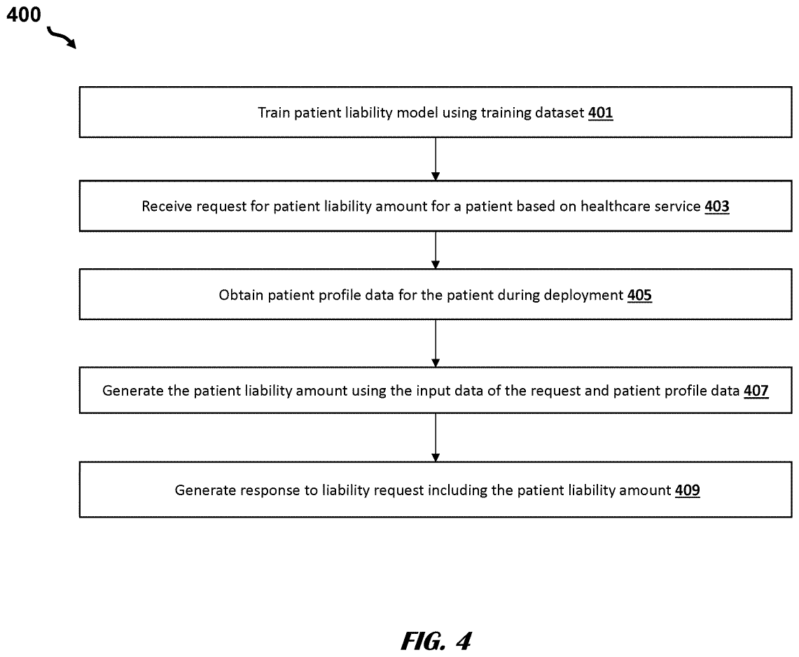

In operation 301, the computer trains one or more machine-learning models of a machine-learning architecture to function as a patient eligibility model using a training dataset. The eligibility model is programmed and trained to predict or determine patient eligibility for a healthcare service, and a patient eligibility engine (or other software program) references and uses to generate an eligibility indicator. The computer obtains (e.g., receives, retrieves) training input data from various types of data sources for a plurality of training patients or training data records. The computer obtains training patient data for the training dataset from various data sources (e.g., third-party servers 111, provider database 103c, analytics database 104). The training data can include structured and unstructured data of various types of data that can influence the determination of whether a patient is eligible for a given healthcare service and, in some embodiments, patient liability for the healthcare service (as in the method 400 of FIG. 4). In some cases, the patient eligibility model is trained using historical data related to patient profiles, insurance claims, and healthcare service records of the provider, among others. This training data includes patient demographics, medical history, insurance coverage details, and previous eligibility determinations. In some examples, the training data can further include details about the healthcare service provided, such as the type of service, the complexity of the procedure, and the associated costs. In at least some examples, the training data can also capture historical billing information, including past claims, payment patterns, and any outstanding balances for training patient records.

The parameters or weights of the layers of the eligibility model are trained on training features or training feature vectors extracted from the patient profile data, where the features or feature vectors may represent certain types of information or data in the patient profile data, such as the patient's insurance coverage, deductible amounts, co-payments, co-insurance percentages, a healthcare service, and a level of complexity of the healthcare service, among others.

In some embodiments, the patient eligibility model uses various machine-learning models or techniques to predict or determine patient eligibility for healthcare services. These models can include decision trees, random forests, support vector machines, and neural networks. For example, a decision tree model can classify patients based on their likelihood of being eligible for a specific healthcare service by analyzing their demographic and medical information. A neural network model can analyze complex patterns in the data to predict eligibility with higher accuracy. The model processes the input data, extracts relevant features, and generates an eligibility indicator, which is then used to inform healthcare providers and patients about the patient's eligibility status. The model continuously learns and improves over time by incorporating new data and feedback from healthcare providers.

In some cases, the computer implements supervised learning to train the eligibility model to recognize patterns and relationships within the data that indicate patient eligibility for healthcare services. The training process involves backpropagation, where the parameters or weights of the eligibility model are updated based on the gradient of the loss function with respect to the eligibility model's parameters. For example, a logistic regression model can be used to predict eligibility based on patient profile data, such as age, medical conditions, and insurance plan details. The eligibility model may be trained by minimizing a loss function, such as cross-entropy loss, through backpropagation and optimization algorithms, such as gradient descent.

For example, the eligibility model may generate predicted outputs for training data inputs. A loss function determines a level of error or loss based upon a difference or similarity between the predicted outputs and expected outputs, as indicated by training labels, user feedback inputs, or actual observed data. The computer executes the loss function to iteratively train the eligibility model using backpropagation to adjust the weights or parameters of the eligibility model to reduce the loss. The computer determines that the eligibility model is trained when the loss satisfies a training threshold.

In operation 303, the computer obtains (e.g., receives, retrieves) patient profile data of a patient during deployment to generate a patient eligibility indicator. As an example, a patient arrives at a healthcare facility and checks in using a kiosk or mobile application. In this example, the patient is prompted to enter insurance information, which the patient enters into the user interface or may be scanned by a scanner capturing an image of an insurance card. The insurance information may include, for example, an insurance provider, policy number, and coverage information. In some examples, the patient may also provide additional information, such as their demographics and medical history. The computer processes this input data and sends a request to the eligibility model to generate the estimated eligibility amount for the patient's healthcare service. The request includes, for example, the patient's insurance information, demographics, and details about the healthcare service.

In operation 305, the computer obtains (e.g., receives, retrieves) patient profile data of a patient during deployment to generate a patient eligibility indicator. The computer obtains the patient data from the various types of data sources, including stored patient data in one or more databases and user-supplied data entered at one or more devices (e.g., provider computing device 103a, kiosk device 103d, patient devices 106). The computer may also retrieve data from insurance information from the one or more databases (e.g., provider database 103c, analytics database 104, third-party servers 111), which contain detailed information about insurance plans, coverage, and patient eligibility for the particular patient, and billing information from the one or more databases (e.g., third-party servers 111, provider database 103c) to obtain historical billing information and payment history data for the patient. The computer can also obtain the user-supplied data (e.g., patient-supplied data, provider-supplied data) from end-user devices, such as patient check-in kiosks or mobile applications, in which the patient or the provider may enter payment or insurance information.

In operation 307, the computer generates a current eligibility indicator for a current patient using the current input data of the request and the obtained patient profile data for the current patient. The computer executes the trained eligibility model using the input data and the patient profile data, which generates the current patient eligibility indicator according to the trained parameters or weights of the patient eligibility model. The eligibility model processes the input data to generate the patient eligibility indicator by performing operations of, for example, data preprocessing, feature extraction, and predictive modeling, among others.

The eligibility model or other software component of the computer may execute feature extraction operations, where the eligibility model is trained to identify and extract certain features or attributes from the patient profile data. These features may include patient demographics, insurance coverage details, medical history, and the complexity of the healthcare service. The eligibility model may include various layers of a machine-learning model trained for predictive modeling. The predictive modeling operations ingest and feed the previously extracted features vectors into the trained eligibility model.

In some implementations, the computer may perform various normalizing and preprocessing operations on the current input patient data or current extracted features. The computer may feed the input patient data into the eligibility model, which applies the trained parameters or weights on the current input patient data, current extracted input features, current feature vectors of the current patient. The trained eligibility model may execute predictive machine-learning technique and operations, such as regression analysis, decision trees, or neural networks using the extracted feature vectors.

As an example, the eligibility model can predict a patient's eligibility for a healthcare service by first extracting a current feature vector from the current patient data of the current patient. The feature vector is a numerical representation of various attributes related to the patient, such as age, gender, medical history, insurance coverage details, and previous healthcare service usage. Each attribute in the feature vector corresponds to a specific aspect of the patient profile that is relevant to determining eligibility. In this example, the eligibility model processes the current feature vector through a series of layers in a neural network. An input layer receives the feature vector, and each subsequent layer applies a set of weights and activation functions to transform the input data. The trained hidden layers of the neural network represent relationships within the features. In some cases, the eligibility model uses a logistic regression layer as the final output layer to generate an eligibility score indicating a likelihood of the patient being eligible for the healthcare service. The probability score is compared against a predefined threshold to determine the final eligibility status. If the score exceeds the threshold, the patient is predicted to be eligible; otherwise, the patient is predicted to be ineligible.

In another example, the eligibility model can use a decision tree to predict eligibility. The decision tree splits the feature vector into branches based on specific criteria, such as age or insurance coverage. Each branch represents a decision point, and the model follows the branches to reach a final decision node that indicates the patient's eligibility status. The decision tree is trained using historical data, where each branch and decision point is optimized to accurately classify patients based on eligibility.

In operation 309, the computer generates a response containing the patient eligibility indicator. The computer transmits the response to a client computer (e.g., provider computing device 103a, patient devices 106) for display to an end-user via a user interface. Additionally or alternatively, the computer provides the eligibility indicator as a machine-readable output that can be ingested as an input to one or more downstream operations, such as a liability model for determining a patient liability amount for the particular healthcare service (e.g., method 400 of FIG. 4).

### Estimating Patient Liability for Treatments

FIG. 4 shows a flowchart of operations of a computer-implemented method 400 for generating an estimated patient liability using one or more machine-learning models of a machine-learning architecture. A computing device (e.g., analytics server 102, provider server 103b) executes software programming (e.g., provider management app 120) of a machine-learning model (e.g., patient estimate model) for ingesting various types of patient-related data from various data sources, determining healthcare services available to the patient, and generating the estimated patient liability amount. For ease of description and understanding, the operations of the computer-implemented method 400 are described as being performed by a computer, though embodiments may be implemented using any type of computing device and/or may be implemented using multiple computing devices.

In operation 401, the computer trains one or more machine-learning models as a patient liability model using a training dataset, which includes a machine-learning model programmed and trained to generate an estimated patient liability amount. The computer obtains (e.g., receives, retrieves) training input data from various types of data sources for a plurality of training patients or training data records. The training data can include structured and unstructured data of various types of data that can influence the estimation of patient liability. The training data can include patient demographics such as age, gender, and medical history. The training data can also include insurance information, including the type of insurance plan, deductible amounts, co-payments, and co-insurance percentages. In some examples, the training data can further include details about the healthcare service provided, such as the type of service, the complexity of the procedure, and the associated costs. The training data can also include historical billing information, including past claims, payment patterns, and any outstanding balances for training patient records.

The computer trains the liability model to determine patient liability amounts using patient profile data that includes various types of patient profile data, such as financial responsibility for healthcare services. In some implementations, the computer trains the machine-learning models to determine patient eligibility for the healthcare service (as in the method 300 of FIG. 3). The computer obtains training patient data for the training dataset from various data sources (e.g., third-party servers 111, provider database 103c, analytics database 104), such as patient demographics, insurance information, healthcare service details, and historical billing information, among others. The training data sources may include training EHR data for training patients, training financial data for training patients, and training insurance data, containing information about potential insurance plans, coverage, and training patient eligibility.

The parameters or weights of the layers of the liability model are trained on training features or training feature vectors extracted from the patient profile data, where the features or feature vectors may represent certain types of information or data in the patient profile data, such as the patient's insurance coverage, deductible amounts, co-payments, co-insurance percentages, a healthcare service, and a level of complexity of the healthcare service, among others.

The computer may feed the training data into the liability model and executes various training operations, including back propagation, loss functions, and optimization algorithms, such as stochastic gradient descent, in order to adjust the parameters or weights of the liability model and minimize a level of error or loss between predicted outputs of the liability model and expected outputs (e.g., the actual training data indicated by training labels). In an example, the liability model is trained to recognize patterns and relationships within the data that indicate patient liability for healthcare services. The training process involves backpropagation, where the parameters or weights of the liability model are updated based on the gradient of the loss function with respect to the liability model's parameters.

In some embodiments, the patient liability model uses various machine-learning models or techniques to predict or determine the patient liability amount for healthcare services. These models can include decision trees, random forests, support vector machines, and neural networks. The liability model is trained to ingest the input data, extract relevant features, and generate the liability amount, which is then used to inform healthcare providers and patients about the patient financial responsibility. The liability model continuously learns and improves over time by incorporating new data and feedback from healthcare providers.

For example, the liability model may generate predicted outputs for training data inputs. A loss function determines a level of error or loss based upon a difference or similarity between the predicted outputs and expected outputs, as indicated by training labels, user feedback inputs, or actual observed data. The computer executes the loss function to iteratively train the liability model using backpropagation to adjust the weights or parameters of the liability model to reduce the loss. The computer determines that the liability model is trained when the loss satisfies a training threshold.

In operation 403, the computer receives a request for the liability model to generate an estimated liability amount for a patient. As in a previous example, a patient arrives at a healthcare facility and checks in using a kiosk or mobile application. In this example, the patient is prompted to enter insurance information, which the patient enters into the user interface or may be scanned by a scanner capturing an image of an insurance card. The insurance information may include, for example, an insurance provider, policy number, and coverage information. In some examples, the patient may also provide additional information, such as their demographics and medical history. The computer processes this input data and sends a request to the liability model to generate the estimated liability amount for the patient's healthcare service. The request includes, for example, the patient's insurance information, demographics, and details about the healthcare service.

In operation 405, the computer obtains (e.g., receives, retrieves) patient profile data of a patient during deployment to generate an estimated patient liability. The computer obtains the patient data from various types of data sources, including stored patient data in one or more databases and user-supplied data entered at one or more devices (e.g., provider computing device 103a, kiosk device 103d, patient devices 106). The computer may also retrieve data from insurance information from one or more databases (e.g., provider database 103c, analytics database 104, third-party servers 111), which contain detailed information about insurance plans, coverage, and patient eligibility for the particular patient, and billing information from the one or more databases (e.g., third-party servers 111, provider database 103c) to obtain historical billing information and payment history data for the patient. The computer can also obtain the user-supplied data (e.g., patient-supplied data, provider-supplied data) from end-user devices, such as patient check-in kiosks or mobile applications, in which the patient or the provider may enter payment or insurance information.

In operation 407, the computer generates the patient liability amount for a current patient using the current input data of the request and the obtained patient profile data for the current patient. The computer executes the trained liability model using the input data and the patient profile data, which generates the patient liability amount according to the trained parameters or weights of the patient liability model. The liability model processes the input data to generate an estimated liability amount by performing operations of, for example, data preprocessing, feature extraction, and predictive modeling, among others.

The liability model or other software component of the computer may execute feature extraction operations, where the liability model is trained to identify and extract certain features or attributes from the patient profile data. These features may include patient demographics, insurance coverage details, medical history, and the complexity of the healthcare service. The liability model may include various layers of a machine-learning model trained for predictive modeling. The predictive modeling operations ingest and feed the previously extracted features vectors into the trained liability model.

In some implementations, the computer may perform various normalizing and preprocessing operations on the current input patient data or current extracted features. The computer may feed the input patient data into the liability model, which applies the trained parameters or weights on the current input patient data, current extracted input features, current feature vectors of the current patient. The trained liability model may execute predictive machine-learning technique and operations, such as regression analysis, decision trees, or neural networks using the extracted feature vectors.

As an example, the liability model can predict a patient's liability amount for a healthcare service by first extracting a current feature vector from the current patient data of the patient. The feature vector is a numerical representation of various attributes related to the patient, such as age, gender, medical history, insurance coverage details, and previous healthcare service usage. Each attribute in the feature vector corresponds to a specific aspect of the patient profile that is relevant to determining patient liability amounts. In this example, the liability model processes the current feature vector through a series of layers in a neural network. An input layer receives the feature vector, and each subsequent layer applies a set of weights and activation functions to transform the input data. The trained hidden layers of the neural network represent relationships within the features to compute a predicted liability amount for the current patient for healthcare service. In some cases, the liability model uses a logistic regression layer as the final output layer to generate the patient liability amount for the current patient for the healthcare service.

In another example, the liability model can use a decision tree to predict the patient liability amount. The decision tree splits the feature vector into branches based on specific criteria, such as age or insurance coverage. Each branch represents a decision point, and the liability model follows the branches to reach a final decision node that indicates the likely liability amount. The decision tree is trained using historical data, where each branch and decision point is optimized to accurately classify patients based on prior liability amounts for healthcare services and, in some cases, patient eligibility.

In operation 409, the computer generates a response containing the patient lability amount. The computer transmits the response to a client computer (e.g., provider computing device 103a, patient devices 106) for display to an end-user via a user interface. Additionally or alternatively, the computer provides the patient lability amount as a machine-readable output that can be ingested as an input to one or more downstream operations.

### Third-Party Integration Check (e.g., Credit Check)

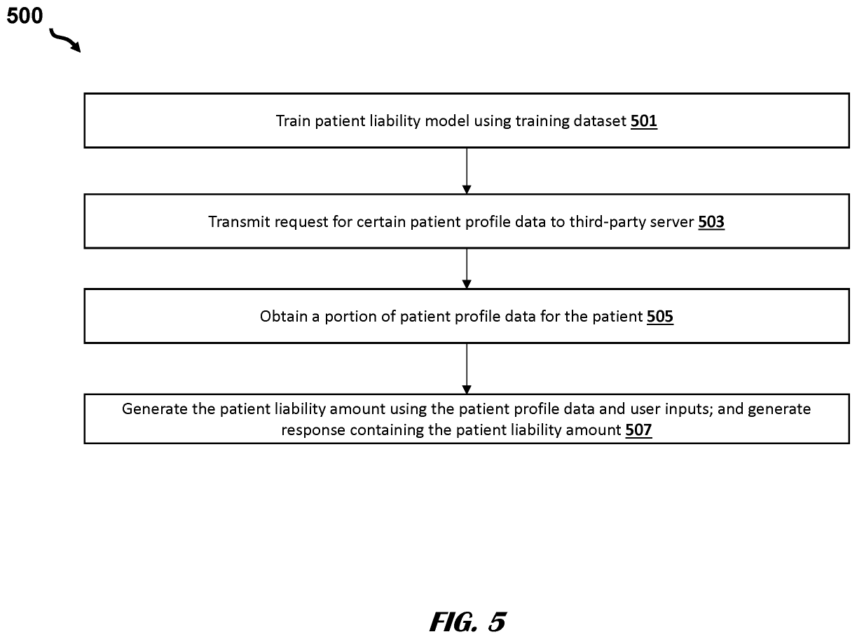

FIG. 5 shows a flowchart of operations of another embodiment for a computer-implemented method 500 for generating an estimated patient liability using a patient liability amount using third-party patient data hosted at a third-party database. A computing device (e.g., analytics server 102, provider server 103b) executes software programming (e.g., provider management app 120) of a machine-learning model (e.g., patient estimate model) for ingesting various types of patient-related data from various data sources, determining healthcare services available to the patient, and generating the estimated patient liability amount. For ease of description and understanding, the operations of the computer-implemented method 500 are described as being performed by a computer, though embodiments may be implemented using any type of computing device and/or may be implemented using multiple computing devices.

In operation 501, the computer trains one or more machine-learning models as a patient eligibility model and/or patient liability model using a training dataset, which includes a machine-learning model programmed and trained to generate an estimated patient liability amount. The training dataset may include training operations as described previously, and the types of data generated or hosted at third-party service and hosted on third-party servers. The training operations and structures of the machine-learning models of the method 500 may implement those previously described and need not be repeated in the description of the method 500.

In operation 503, the computer transmits a request for certain types of patient profile data to a third-party server that hosts the types of patient profile data. As an example, the computer may transmit a request for a credit score of a current patient from third-party server hosting a credit-reporting service as part of the process to predict the patient's eligibility and/or liability amount for a healthcare service.

In operation 505, the computer obtains (e.g., receives, retrieves) patient profile data of a patient during a deployment phase to determine a patient eligibility status or liability amount. The computer uses various types of patient profile data, including the patient profile data received from the third-party server, and any user-supplied inputs from a patient or provider. Additionally, in the ongoing example, the patient data may include the patient's credit score from a credit-reporting service returned in a response from the third-party server.

The computer extracts a current feature or feature vector for the current patient from the patient data, which includes attributes, such as a credit score, age, gender, medical history, insurance coverage details, and previous healthcare service usage, among others.

In operation 507, the computer generates the patient eligibility indicator and/or patient liability amount using the patient profile data and any user input data. The computer generates a message containing the patient eligibility indicator and/or patient liability amount, and transmits the message to an end-user device of a provider or patient.

In some implementations, the machine-learning model processes the updated feature vector through a series of layers in a neural network. The input layer receives the feature vector, and each subsequent layer applies a set of weights and activation functions to transform the input data. The trained hidden layers of the neural network capture complex patterns and relationships within the data, allowing the model to learn from historical examples of patient eligibility determinations or patient liability amounts. The machine-learning model may generate the patient eligibility score indicating a likelihood of the patient being eligible for the healthcare service and/or the patient liability amount for the healthcare service. In some cases, the machine-learning model uses a logistic regression layer as the final output layer to generate the eligibility score and/or the patient liability amount. Optionally, the computer compares the probability score against a predefined threshold to determine the final eligibility status. If the score exceeds the threshold, the patient is predicted to be eligible; otherwise, the patient is predicted to be ineligible

In some implementations, the machine-learning model can use a decision tree to predict eligibility and/or the patient liability amount. The decision tree splits the feature vector into branches based on specific criteria, such as age, insurance coverage, and credit score. Each branch represents a decision point, and the machine-learning model follows the branches to reach a final decision node that indicates the patient's eligibility status and/or patient liability amount. The decision tree is trained using historical data, where each branch and decision point is optimized to accurately classify patients based on eligibility status or liability amount for the healthcare service.

The computer transmits the response to a client computer (e.g., provider computing device 103a, patient devices 106) for display to an end-user via a user interface. Additionally or alternatively, the computer provides the eligibility indicator and/or patient liability amount as a machine-readable output that can be ingested as an input to one or more downstream operations, such as a machine-learning model for determining the patient eligibility and/or patient liability amount for the particular healthcare service (e.g., method 300, method 400).

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, attributes, or memory contents. Information, arguments, attributes, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-Ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A computer-implemented method for identifying automated operations for management systems, the method comprising:
obtaining, by a computer, management operation data for one or more management operation executed by a management application;
for each management operation, extracting, by the computer, a feature vector representing the management operation of the management operation data;
identifying, by the computer, a management operation as a pattern operation based on the feature vector representing the management operation and a number of instances of the management operation satisfying a regularity threshold number of instances;
for each pattern operation, generating, by the computer, a candidate operation score for the pattern operation based upon the feature vector representing one or more features extracted for the management operation identified as the pattern operation and a set of improvement features representing improved management operations; and
generating, by the computer, an instruction for the management application to execute the pattern operation as a candidate automated operation in response to the computer determining that the candidate operation score satisfies an automation threshold.

2. The method according to claim 1, further comprising generating, by the computer, a message indicating the candidate automated operation for display at user interface of a management computing device.

3. The method according to claim 1, further comprising training, by the computer, a candidate automation machine-learning model to generate the candidate operation score.

4. The method according to claim 1, wherein identifying the management operation as the pattern operation includes generating, by the computer, a pattern operation score based on the feature vector and a set of expected pattern features representing one or more expected pattern operation, the pattern operation score representing a likelihood of the management operation being the pattern operation.

5. The method according to claim 4, wherein the computer determines that the candidate operation score satisfies a predefined pattern threshold and the regularity threshold.

6. The method according to claim 4, further comprising training, by the computer, a candidate automation machine-learning model to generate the pattern operation score.

7. The method according to claim 1, wherein the management operation data includes patient profile data obtained by the computer from an external server.

8. A system for identifying automated operations for management systems, the system comprising:
a computer comprising at least one processor, configured to:
obtain, from a database, management operation data for one or more management operations executed by a management application;
for each management operation, extract a feature vector representing the management operation of the management operation data;
identify a management operation as a pattern operation based on the feature vector representing the management operation and a number of instances of the management operation satisfying a regularity threshold number of instances;
for each pattern operation, generate a candidate operation score for the pattern operation based upon the feature vector representing one or more features extracted for the management operation identified as the pattern operation and a set of improvement features representing improved management operations; and
generate an instruction for the management application to execute the pattern operation as a candidate automated operation in response to determining that the candidate operation score satisfies an automation threshold.

9. The system according to claim 8, wherein the computer is further configured to generate a message indicating the candidate automated operation for display at user interface of a management computing device.

10. The system according to claim 8, wherein the computer is further configured to train a candidate automation machine-learning model to generate the candidate operation score.

11. The system according to claim 8, wherein when identifying the management operation as the pattern operation the computer further configured to generate a pattern operation score based on the feature vector and a set of expected pattern features representing one or more expected pattern operations, the pattern operation score representing a likelihood of the management operation being the pattern operation.

12. The system according to claim 11, wherein the computer determines that the candidate operation score satisfies a predefined pattern threshold and the regularity threshold.

13. The system according to claim 11, wherein the computer is further configured to train a candidate automation machine-learning model to generate the pattern operation score.

14. The system according to claim 8, wherein the management operation data includes patient profile data obtained by the computer from an external server.

15. A non-transitory machine-readable storage configured to machine-readable instructions for identifying candidate automated operations for management systems that, when executed by at least one processor, instructs the at least one processor to:
obtain, from a database, management operation data for one or more management operations executed by a management application;
for each management operation, extract a feature vector representing the management operation of the management operation data;
identify a management operation as a pattern operation based on the feature vector representing the management operation and a number of instances of the management operation satisfying a regularity threshold number of instances;
for each pattern operation, generate a candidate operation score for the pattern operation based upon the feature vector representing one or more features extracted for the management operation identified as the pattern operation and a set of improvement features representing improved management operations; and
generate an instruction for the management application to execute the pattern operation as a candidate automated operation in response to determining that the candidate operation score satisfies an automation threshold.

16. The machine-readable storage of claims 15, wherein the instructions further instruct the at least one processor to generate a message indicating the candidate automated operation for display at user interface of a management computing device.

17. The machine-readable storage of claims 15, wherein the instructions further instruct the at least one processor to train a candidate automation machine-learning model to generate the candidate operation score.

18. The machine-readable storage of claims 15, wherein when identifying the management operation as the pattern operation the instructions further instruct the at least one processor to generate a pattern operation score based on the feature vector and a set of expected pattern features representing one or more expected pattern operation, the pattern operation score representing a likelihood of the management operation being the pattern operation.

19. The machine-readable storage of claims 18, wherein the at least one processor determines that the candidate operation score satisfies a predefined pattern threshold and the regularity threshold.

20. The machine-readable storage of claims 15, wherein the instructions instruct the at least one processor to train a candidate automation machine-learning model to generate the pattern operation score.
